(19) United States
(12) Patent Application Publication
(10) Pub. No.: US 2004/0143326 A1
(43) Pub. Date: Jul. 22, 2004

(54) Method of sampling nipple prosthetic devices during breast reconstruction

(76) Inventor: Constance Tizard Holmes, New York, NY (US)

Correspondence Address: Steven Horowitz, Suite 700, 295 Madison Avenue, New York, NY, 10017, US

(21) Appl. No.: 10/374,481

(22) Filed: Feb. 26, 2003

Related U.S. Application Data
(63) Continuation-in-part of application No. 10/349,449, filed on Jan. 22, 2003.

(51) Int. Cl. A61F 2/52

(52) U.S. Cl. 623/7

(57) ABSTRACT

A method of enhancing the third stage of breast reconstruction involves sampling an artificial nipple prosthetic device attachable to a female human breast by trying on and wearing different sized and colored devices at different locations of the breast. This allows the female patient to advise her surgeon where, what size and what color nipple she wants the surgeon to reconstruct. The nipple prosthetic devices have a substantially cylindrical solid nipple portion made of a breathable water-resistant natural or microporous polymeric material and an areola portion surrounding the nipple portion made of the same material. The inside of the device, at both the nipple and areola portions, has a concave shape that conforms to an outer contour of a female human breast and the device is attached by means of a water resistant adhesive layer. The shades of color of the nipple and areola portions conform to the female anatomy.

This application is a continuation in-part of U.S. patent application Ser. No. 10/349,449 for a “Female Nipple Prosthetic Device” filed Jan. 22, 2003 by the same inventor/applicant, Constance Tizard Holmes, which parent application is pending and incorporated herein by reference.

### FIELD OF THE INVENTION

The field of this invention is methods of enhancing breast reconstruction surgery for humans, and more particularly, enhancing the third stage of such surgery, which involves the female nipple.

### BACKGROUND OF THE INVENTION AND DISCUSSION OF THE PRIOR ART

Medically, female breast reconstruction generally occurs in three stages. The first stage is the mastectomy. The second stage involves internal reconstruction of the breast. The third stage involves nipple reconstruction. During the period of time from the mastectomy to the nipple reconstruction the female patient can live without one or both nipples for a period of up to two years. If a pregnancy occurs, it could delay it even further.

Presently, the surgeon typically advises the patient perhaps a day before the nipple reconstruction surgery to draw on her own body a nipple indicating the location and size of the nipple that she would prefer to have the surgeon reconstruct during the third stage surgery. That crude and rushed procedure hardly provides the patient the time or the circumstances for appropriate psychological adjustment in order to make such a decision. It would be quite beneficial to have some way of greatly enhancing the psychological and practical factors surrounding the decision by the patient as to where, how large and what color her reconstructed nipple should be.

Furthermore, the clear majority of breast reconstruction patients never even reach the stage three reconstruction surgery simply due to the difficult psychological toll that the process takes. Accordingly, having some way of greatly enhancing the practical and psychological aspects of the decision by the patient as to where, how large and what color the reconstructed nipple should be would thus also have the incidental but crucial advantage of dramatically increasing the success rate of the entire three stage breast reconstruction procedure.

In order to finally decide on the desired size, location and color of the nipple the patient will receive during the third stage involving nipple reconstruction, it would be very helpful for the female patient to be able to wear and try out for selected periods of time in real life situations a variety of nipple prosthetic devices that simulate a nipple that the surgeon would reconstruct during the third stage of breast reconstruction in accordance with the patient's instructions. The variety of such prosthetic devices would differ in terms of size, location and color—or in terms of other characteristics. In this way, when the surgeon asks the patient to decide on the location, size and color of the nipple she desires, the patient will have had a meaningful opportunity to make that decision, having sampled the product and its location.

The present invention provides a method of using a female nipple prosthetic device by the patient after the masectomy and prior to the nipple reconstruction to enhance the third stage of breast reconstruction, i.e. nipple reconstruction, and to assist the female patient in adjusting to breast reconstruction. As explained, the patient benefits greatly from using an artificial nipple prior to the actual nipple reconstruction. This is true provided the nipple prosthetic devices sampled are suitable for and specifically designed to be worn by a female patient during the second stage of breast reconstruction.

During the second stage of breast reconstruction the patient's skin in the area of the nipple is traumatized and scarred from being aggravated by the implantation procedures of this second stage. Hence, the most appropriate female nipple prosthetic device for the method of the present invention would be one that causes no further irritability or aggravation to the skin in the affected area. This requirement means that the prosthetic device used in the method of the present invention must be breathable and comfortable. Any prosthetic device held against the skin that is not porous is likely to lock in moisture from perspiration. This could cause discomfort and may aggravate and/or irritate the skin. In addition, it could reduce proper blood circulation in that area.

It would be advantageous for the method of the present invention if the nipple prosthetic devices to be sampled could be manufactured simply, preferably out of a single piece of material, and can be available in a variety of sizes, shapes and colors since different women may have different nipples in terms of size, shape and color and hence may prefer different artificial nipples.

Furthermore, the fact that the period of use can be as long as two years and that the nipple can be worn for many days at a time, if not for entire weeks at a time, is an additional reason mandating that the artificial nipple used for the method of the present invention be not only attractive and perceived as authentic but also very comfortable.

To applicant's knowledge, there are no known prior art methods of using nipple prosthetic devices to enhance nipple reconstruction surgery or enhance adjustment to breast reconstruction. In fact, in the past, use of nipple prosthetic devices have been limited to cosmetic and related purposes. Such prosthetic devices used for cosmetic purposes have in fact been made of plastic or other non-breathable materials, which is unsuitable for enhancing and allowing a female patient to adjust to nipple reconstruction surgery. See for example U.S. Pat. No. 5,171,321 to Davis. When worn for relatively short periods of time for cosmetic purposes, plastic may seem to be an appropriate material for an artificial nipple since it is resilient and can be textured to achieve the tactile sensation that simulates a medically real nipple. A prosthetic device worn to enhance breast reconstruction in contrast must be something suitable to be placed on already scarred skin tissue which cannot withstand irritation. Furthermore, such a device replaces a part of the human anatomy and should be manuafactured so that it can be worn for lengthy periods of time if necessary yet be removable if such is desired. For such purposes, plastic and other non-breathable materials are not suitable.

As explained, nipple prosthetic devices used for cosmetic purposes have been made of non-porous plastic and are not suitable for use during breast reconstruction. Even the plastic ones that have been fenestrated with one or more holes are not suitable either. Typically, the placement of a single hole occurs either in the center of the artificial nipple or in its surrounding area, such surrounding area being an artificial areola. It is questionable whether a single hole could alleviate the overall lack of breathability in the surface of the device that contacts the skin.

Furthermore, even a plastic nipple prosthetic device that is fenestrated with multiple holes would not be suitable for use during second stage breast reconstruction. The multiple holes (or for that matter even a single hole) would simply detract from the visual and tactile simulation purpose of the device. The purposes of having an artificial nipple is to simulate a biological nipple from a visual point of view and to simulate the biological nipple from a tactile point of view. Tactile simulation of a real nipple is necessary for any situation in which the nipple would likely be touched, such as during intimate contact. Having holes at various portions of the artificial nipple contradicts both the objective of visual simulation of the nipple as well as the objective of tactile simulation of the real nipple. A medically real nipple does not have holes, does not appear to have holes and not feel like it has holes.

It is also noted that women may be sensitive about the appearance of physical irregularities in their nipple and hence may even exaggerate the significance of an otherwise minor irregularity in the nipple. Accordingly, holes in the nipple, however minute, may be a disadvantage for these women.

In sum, these prior art nipple prosthetic devices used for cosmetic purposes are not suitable to be used to sample nipple prosthetic device in order to enhance the third stage of breast reconstruction. Such devices need to be both very comfortable to wear for long periods of time during the second stage of breast reconstruction or otherwise and be as perfectly as possible identical to a biological female human nipple both visually and in terms of touch. Such prosthetic devices, in order to be suitable for being tried on and sampled by the female patient must be breathable and should not have holes that are perceptible either visually or through touch.

### SUMMARY OF THE PRESENT INVENTION

A method of enhancing the third stage of breast reconstruction involves sampling an artificial nipple prosthetic device attachable to a female human breast by trying on and wearing different sized and colored devices at different locations of the breast. This allows the female patient to advise her surgeon where, what size and what color nipple she wants the sureon to reconstruct. The nipple prosthetic devices have a substantially cylindrical solid nipple portion made of a breathable water-resistant natural or microporous polymeric material and an areola portion surrounding the nipple portion made of the same material. The inside of the device, at both the nipple and areola portions, has a concave shape that conforms to an outer contour of a female human breast and the device is attached by means of a water resistant adhesive layer. The shades of color of the nipple and areola portions conform to the female anatomy.

### IMPORTANT OBJECTS AND ADVANTAGES

The following important objects and advantages of the present invention are:

(1) to provide a method of enhancing breast reconstruction surgery for human females;

(2) to provide such a method that involves allowing a woman to sample and in a very practical manner decide on a set of physical characteristics of her desired nipple prior to nipple reconstruction surgery, including but not limited to size, color, shape, weight, porosity, rigidity, breathability and location;

(3) to provide such a method that involves allowing a woman to decide on the size, location and color of her desired nipple prior to nipple reconstruction surgery;

(4) to provide a method of enhancing third stage breast construction by allowing the female patient to respond in a confident and meaningful way to the surgeon's question as to where that patient desires to have her nipple reconstruction and with what size and color nipple;

(5) to provide such a method that involves samples a variety of nipple prosthetic devices;

(6) to provide such a method that involves samples nipple prosthetic devices for long periods of time in real life situations including while taking a shower and/or while swimming;

(7) to provide a method of enhancing breast reconstruction by sampling nipple prosthetic devices that are breathable and can be worn comfortably for days at a time;

(8) to provide a method of enhancing nipple reconstruction by sampling nipple prosthetic devices that simulate a medically real female nipple both visually and in terms of tactile perception;

(9) to provide a method of enhancing nipple reconstruction surgery by sampling nipple prosthetic devices that simulate both the nipple and the surrounding areola;

(10) to provide a method of enhancing nipple reconstruction surgery by sampling nipple prosthetic devices that are either made of breathable cotton or another natural breathable loosely woven fibrous material or are made from microporous polymeric material;

(11) to provide a method of enhancing breast reconstruction surgery by sampling nipple prosthetic devices that are resilient and that can be easily manufactured in a wide variety of sizes, shapes and colors;

(12) to provide a method of enhancing breast reconstruction surgery by sampling nipple prosthetic devices that can be used by a woman undergoing breast reconstruction to try out the size, location and color of a nipple; and

(13) to provide a method of enhancing breast reconstruction surgery by sampling nipple prosthetic devices that are water resistant in that they can be worn while showering, swimming or similar activities without the device falling off or being damaged.

### BRIEF DESCRIPTION OF THE DRAWINGS

FIG. 1 is a side elevational view of an example of a device used in the method of the present invention while attached to a female breast;

FIG. 2 is a cross-sectional view of such a device used in the method of the present invention;

FIG. 3 is a top plan view of the device used in the method of the present invention including the peelable backing.

### DETAILED DESCRIPTION OF THE PREFERRED EMBODIMENT

The method of the present invention is a method by which a female patient enhances a third stage of her breast reconstruction by sampling nipple prosthetic devices during the second stage of the female patient's breast reconstruction so that she can advise her surgeon what color, size (and other characteristics) and location her nipple should be.

The female patient

(a) selects a first candidate nipple prosthetic device that has a particular size and color,

(b) attaches the first candidate nipple prosthetic device to her breast that is undergoing reconstruction at an initial location of her choosing by means of an adhesive strong enough so that the first candidate nipple prosthetic device (and subsequent candidate nipple prosthetic devices) stays on the breast during a wearing of the device but weak enough to permit easy removal of said device,

(c) wears said first candidate nipple prosthetic device at the initial location for a period of time including in as many real life situations as she desires, said period of time optionally including time gaps of non-use, said first candidate nipple prosthetic device being breathable and water-resistant;

(d) removes the first candidate nipple prosthetic device from the initial location,

(e) repeats steps (b) through (d) for the first candidate nipple prosthetic device at as many other locations on said breast as she desires,

(f) repeats steps (a) through (e) for as many subsequent candidate nipple prosthetic devices having a different size and/or shape from a just previously sampled candidate nipple prosthetic device as desired until the female patient satisfactorily decides on a final size, color and location of a desired nipple.

The method of the present invention contemplates that it may very well be advantageous for medical reasons to allow the tissue on the patient's skin to rest during varying periods of time, such as overnight, in between applications of the device. Hence, a patient may choose to re-apply a selected nipple prosthetic device of identical characteristics to the same location the next morning after an unspecified period of non-use. However, in a preferred embodiment the patient would be re-applying a different specimen of the selected nipple prosthetic device, i.e. one having the same characteristics, rather than re-applying the very same selected device. Once peeled off the backing the actual identical device would not be re-used, preferably.

Accordingly, this method as described is intended to also include situations where the patient chooses to put the selected nipple prosthetic device back (preferably this means another specimen of the device having the same selected characteristics) to essentially the same location on her breast that she previously wore the device after having removed it from that location. She may simply want to try it out again at that location after a rest. Accordingly, therefore, the term “first candidate nipple prosthetic device” and the term “subsequent candidate nipple prosthetic device” refer to all specimens of a selected device having the same set of physical characteristics.

Moreover, it is understood that the term “other locations” in step (e) of the method of the present invention shall include replacement at an identical (or essentially identical) location on other occasion. It is the operating assumption of the method of the present invention that it is technically impossible to re-place a nipple prosthetic device at precisely the same location as before. Hence, for the purposes of the method of the present invention, any re-adhering of the same device to an identical or near identical location would still be considered a different or “other” location the next time it is put there. This is true regardless of whether in the interim there was a placement of the device at a completely different location.

It should also be understood that the method of the present invention has been described in terms of sampling nipple prosthetic devices of varying size, color and location to finally decide on their size, location and color. However, the present invention also contemplates other variables besides the three mentioned. For example, the method includes the possibility of having the patient sample candidate nipple prosthetic devices of varying size, color and shape at different locations also. The method of the present invention also contemplates sampling and deciding on other variables, such as the weight of the device, its porosity, breathability, rigidity. Furthermore, any other structural or functional characteristic of the device used in the method of the present invention are likewise contemplated alone or in combination with the size, color, location, shape, rigidity, porosity, weight described.

Accordingly, the following general method is contemplated:

The female patient, during the second stage of her breast reconstruction,

(a) selects a first candidate nipple prosthetic device that has a particular set of physical characteristics, such characteristics include size, shape, color, porosity, breathability, weight, rigidity.

(b) attaches the first candidate nipple prosthetic device to her breast that is undergoing reconstruction at an initial location of her choosing by means of an adhesive strong enough so that the first candidate nipple prosthetic device (and subsequent candidate nipple prosthetic devices) stays on the breast during a wearing of the device but weak enough to permit easy removal of said device,

(c) wears said first candidate nipple prosthetic device at the initial location for a period of time including in as many real life situations as she desires, said period of time optionally including time gaps of non-use, said first candidate nipple prosthetic device being breathable and water-resistant;

(d) removes the first candidate nipple prosthetic device from the initial location,

(e) repeats steps (b) through (d) for the first candidate nipple prosthetic device at as many other locations on said breast as she desires,

(f) repeats steps (a) through (e) for as many subsequent candidate nipple prosthetic devices having a different set of characteristics from a just previously sampled candidate nipple prosthetic device as desired until the female patient satisfactorily decides on a final set of characteristics of a desired nipple and its location.

Even though steps (a) through (d) are characterized in terms of a first candidate nipple prosthetic device, it should be understood that the repetition of these steps in step (e) and in step (f) is also for candidate nipple prosthetic devices that are subsequent to the first—i.e. the second, third, etc. It is further noted that the description of the steps of the method of the present invention is also not intended to preclude a female patient from going back to a previously selected first or subsequent candidate nipple prosthetic device at any point in the selection process. Hence the repetition of steps (a) through (d) in steps (e) and (f) can actually even be for the re-wearing of the first candidate nipple prosthetic device after some number of subsequent candidate nipple prosthetic devices have been worn.

It is also noted that a nipple prosthetic device can be said to have a “different set of characteristics” from a previously sampled nipple prosthetic device if even only one of the characteristics of the device differs.

It is further noted that in step (c) of the method of the present invention the period of time can include time gaps of non-use that occur at the end of said period of time so that there could for example be a time gap of non-use between the wearing of the first candidate nipple prosthetic device and a subsequent nipple prosthetic device or between the wearing of two different subsequent nipple prosthetic devices.

It is further noted that other methods of carrying out step (b) are contemplated by the present method. That is, other methods of attaching the candidate nipple prosthetic device to the breast are contemplated other than a layer of adhesive on the nipple prosthetic device. These include applying adhesive that does not form a part of the nipple prosthetic device to the nipple prosthetic device in order to attach that device to the breast. These also include using other means of attachment besides traditional glue-type adhesive, namely using static electricity, using a frictional fit and using any other suitable effective way to temporarily and removably cause said candidate nipple prosthetic device to adhere to the breast. Accordingly, the term “adhesive”, when used in the claims to denote adhesive used to attach the candidate nipple prosthetic device to the breast, shall be understood as including anything that adheres temporarily and removably so that the device does not fall off when worn yet can be easily removed. Furthermore, in some embodiments where traditional glue-type adhesive is used, the device includes a layer of adhesive as part of said device; in other embodiments the device does not.

Accordingly, the method of the present invention is intended to and is able to cover any process of selecting, attaching and wearing different nipple prosthetic devices to decide on a desired location and set of physical characteristics of a nipple for breast reconstruction.

To further illustrate the method of the present invention, the method of the present invention will now be illustrated by reference to nipple prosthetic devices that are illustrated in the accompanying drawings, which describe a device to be used by female patients to sample the size, location and color (or other physical characteristics such as shape, rigidity, porosity, weight, etc.) of their desired nipple. The nipple prosthetic device illustrated as being used in the method of the present invention has been assigned reference numeral 10 Other elements have been assigned the reference numerals referred to below.

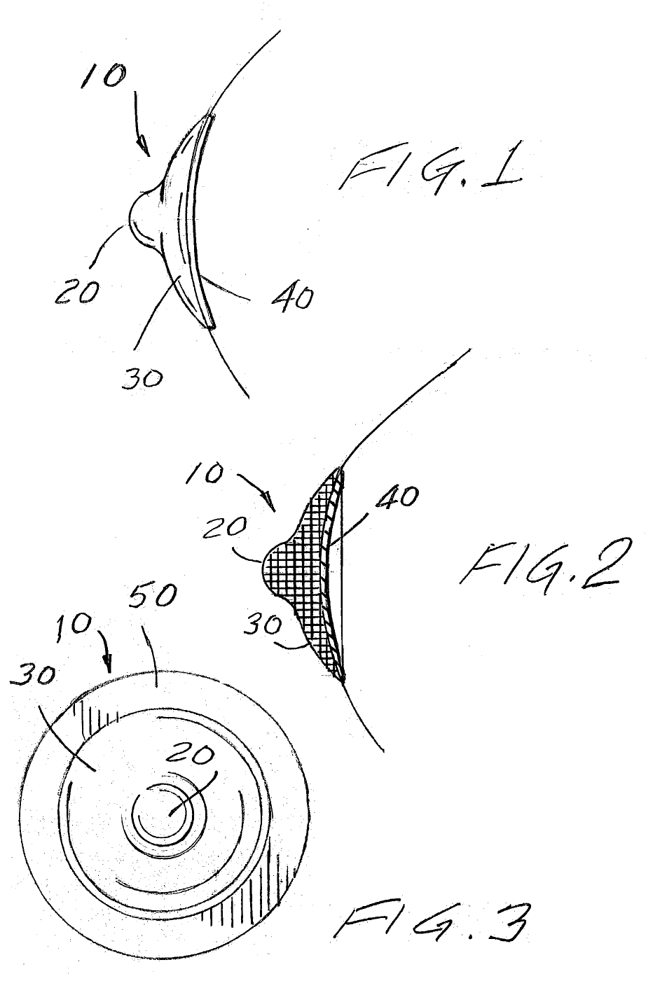

As seen from FIGS. 1-3, nipple prosthetic device 10 is shown as comprising a nipple portion 20 and an areola portion 30. Nipple prosthetic device 10 is thus attachable to a female human breast, usually one which has undergone breast reconstruction, by means of adhesive layer 40. Device 10 is stored against a peelable backing 50 (in embodiments where device 10 is defined to include backing 50 then the remainder of device 10 is stored against peelable backing 50). Typically, peelable backing 50 may be latex or other suitable material. Peelable backing 50 may be made of any material known to those skilled in the art to be suitable for removable contact with nipple portion 20 and areola portion 30, i.e. with breathable natural loosely woven fibrous material having a layer of adhesive under it. Peelable backing 50 allows the remainder of device 10 to be peeled off prior to the step of attaching the device to the breast during the method of the present invention. Since the inside of device 10 is concave, peelable backing 50 is also concave. Alternatively, peelable backing would be in actual contact only with areola portion 30 or with an outer rim portion of areola portion 30.

As seen from FIGS. 1-2, nipple portion 20 and areola portion 30 may be described as substantially bell shaped when viewed together and from the side since said portions are shaped to replicate a biological female human breast nipple. Arguably, nipple portion 20 alone may also be described as substantially cylindrical overall.

Nipple portion 20 and areola portion 30 are preferably formed from one continuous piece of material so these portions are made from the same material. Besides the shape, a difference between the nipple portion 20 and areola portion 30 is, however, observable in that there is a gradual change in color from lighter to darker as one goes from nipple portion 20 to areola portion 30. The preferred color of nipple portion 20 and areola portion 30 is the color of a natural female nipple and areola, which may be a shade of beige or yellow-reddish or skin color.

It is an important feature of the present invention that nipple portion 20 and areola portion 30 of nipple prosthetic device 10 used in the method of the present invention are made of a breathable material. Preferably, it is also a natural material to minimize or avoid allergic skin reactions. Furthermore, nipple portion 20 is not hollow but is solid so as to prevent collapse of nipple portion 20. Typically, the material from which nipple portion 20 and areola portion 30 are made is natural material rather than plastic or other man-made material. Preferably, the material used for nipple portion 20 and areola portion 30 should not be natural rubber, synthetic rubber, silicone or any plastic including but not limited to polyurethane, polyethylene, polypropylene or another synthetic polymer or plastic since these are not natural breathable materials. Rather the material should be cotton or another loosely woven fibrous material that is breathable. Ideally, the material should also be downy, i.e. soft.

In an alternative embodiment, the material from which nipple portion 20 and areola portion 30 are made is a water-resistant and breathable man-made material such as the polytetrafluorethylene disclosed in U.S. Pat. No. 4,187,390. Similarly, it is contemplated by the present invention that certain microporous man-made polymeric materials can be breathable and water-resistant and be usable as the material for the nipple portion 20 and areola portion 30.

It should be understood that the term “loosely woven” should exclude material that is so loosely woven that it either has holes that are visible or perceptible through touch or that the resilience or durability of the material are compromised with respect to the purposes of the nipple prosthetic device of the present invention, namely accepting an adhesive layer and being able to adhere to the breast and stay there without being damaged, deformed or knocked off. The texture of the material used for nipple portion 20 and areola portion 30 should not be too loosely woven device 10 must be sufficiently resilient and durable and maintain its shape in the face of forceful contact.

For example, certain kinds of loosely woven cotton are used to make gauze bandages and are breathable and suitable for nipple portion 20 and areola portion 30. Certain bandages contain the breathable material that represent examples of the desired material. Furthermore, such bandage materials are ideally suited to hold a layer of adhesive underneath that is water-resistant. Purely by way of example, an adhesive dressing bandage sold under the brand name “Coverlet” and which is manufactured by a company called Belersdorf-Jobst, Inc. of Rutherford College, North Carolina 28671 is believed to be breathable and water-resistant. The Coverlet brand bandages, though understood to be breathable, are dense enough to preclude the appearance of holes. This is in contrast to the breathable natural material of Sheer Band-Aid brand bandages sold by Johnson & Johnson, which though breathable has readily visible holes, or the Plastic Band-Aid brand bandages of Johnson & Johnson, which is not breathable. Other brands of breathable natural material or other breathable loosely woven fibrous material are well known to those skilled in the art and are available to satisfy the requirements of being breathable material yet having no visible holes.

However, since the breathable material from which nipple portion 20 and areola portion 30 are made must be shaped to duplicate the shape of a female nipple and areola, as shown in FIGS. 1-3, the thin layer used in a thin breathable bandage for wounds would alone not be suitable. Rather a thicker version of the same breathable material would have to be utilized and in addition shaped to conform to the shape of a female human breast nipple and the surrounding areola.

The areola portion 30 has a substantially round outer perimeter and surrounds nipple portion 20. Furthermore, the inside of device 10 has a concave shape at the inside surface of both nipple portion 20 and areola portion 30 so as to conform to an outer contour of a female human breast. It should be understood that nipple portion 20 includes a surface that is adjacent to adhesive layer 40 (in the embodiments where adhesive layer 40 is not limited to being in contact with areola portion 30). Upon attachment of the nipple prosthetic device 10 to the female breast, nipple portion 20 projects outwardly from an outer surface of the female human breast. In a preferred embodiment, nipple portion 20 projects substantially perpendicularly outward from said outer surface of said female human breast. By “substantially perpendicularly” is meant that an imaginary axis running longitudinally through a center of the nipple portion 20 would intersect a line tangent to the curved surface of the breast at an angle that deviates by not more than approximately twenty degrees from the normal.

Underneath nipple portion 20 and areola portion 30 is adhesive layer 40, a thin layer of adhesive whose effectiveness and/or adhering strength is specifically designed to be largely unaffected by water such that device 10 is water-resistant. Furthermore, nipple portion 20 and areola portion 30 remain breathable even though they are in contact with adhesive layer 40 either because the adhesive of adhesive layer 40 is not universally applied on the entire inside relevant surface of device 10 and/or because adhesive 40 is only lightly applied just enough to create nonpermanent adherence without disturbing breathability and/or because of other reasons known to those skilled in the art.

In one embodiment, adhesive layer 40 covers an area that is adjacent to the entire inside surface of nipple portion 20 and areola portion 30. Alternatively, adhesive layer 40 is situated so as not to be in contact with nipple portion 20 at all (or in other alternative embodiments so as not to be in contact with all of nipple portion 20) but rather covers an area that is adjacent to and in contact with the inside concave surface of areola portion 30 and in fact contacts the entire such inside concave surface of areola portion 30. In a further alternative, adhesive layer 40 is situated so as to cover an area that is adjacent to and in contact with only a continuous portion of the inside surface of areola portion 30, and preferably a continuous annular segment of areola portion 40. In a further alternative embodiment, adhesive layer 40 is situated so as to cover an area that is adjacent to and in contact with only a noncontinuous segment of areola portion. Alternatively, adhesive layer 40 is situated so as to be in contact with all or a portion of nipple portion 20 but not any of areola portion 30. In any of the alternative embodiments and in the preferred embodiment also, the adhesive layer 40 must cover a sufficient surface area so as to generate the required adhesion for device 10 to stay immediately adjacent the female reconstructed breast for long uninterrupted periods of time, including in or in exposure to water.

Furthermore, since the color, shape and size of female nipples vary, device 10 can be molded into different sizes and different colors. The different shapes of device 10 would arise from the nipple portion 20 of said device being shaped differently. The nipple portion 20 shown in FIGS. 1-2 is merely illustrative of one such shape. Device 10 would be available in a variety of shapes, sizes and colors.

In order to conform to the female anatomy, the nipple and areola portions are colored to conform to an actual female human nipple and areola and wherein a color of the nipple portion gradually changes to a darker color of the areola portion in an area of the areola portion closest to the nipple portion.

With respect to the sizes and colors of the nipple prosthetic devices sampled by the female patient, in a preferred embodiment the sizes of the nipple portion would range in diameter from one quarter of an inch to one half of an inch and would have increments of one sixteenth of an inch in diameter. The diameters of the areola portions and of the overall device would range from one inch to two inches correspondingly. All size descriptions in this application are approximations. Accordingly, the smallest version would be a prosthetic device of approximately one inch in diameter having a nipple portion diameter of approximately one quarter inch in diameter. The next smallest version would be a prosthetic device of approximately one and one quarter inch in diameter and would have a nipple portion diameter of approximately five sixteenths of an inch. The largest would be a prosthetic device having an overall diameter of approximately two inches and a nipple portion diameter of approximately half an inch. Although these are the expected sizes, the present invention contemplates a method that samples nipple prosthetic devices of other sizes as well.

As a result of the above description of the candidate nipple prosthetic devices, it should be apparent that the method described above of the present invention can be modified to state further details. For example, selecting the first and subsequent candidate nipple prosthetic devices may involve selecting candidate nipple prosthetic devices that contain a nipple portion and an areola portion. In addition, attaching the first and subsequent candidate nipple prosthetic devices may involve attaching candidate nipple prosthetic devices wherein the adhesive layer covers an area adjacent to and in contact with at least part of an inside surface of the candidate nipple prosthetic device sufficient to adhere said candidate nipple prosthetic device to the female breast, the areola portion of the candidate nipple prosthetic device surrounding said nipple portion of said candidate nipple prosthetic device and having a concave shape that conforms to an outer contour of a female human breast, said nipple portion aligned with said areola portion so that upon attachment of the candidate nipple prosthetic device to said breast, said nipple portion projecting outwardly from an outer surface of said breast.

Similarly, selecting the first and subsequent candidate nipple prosthetic devices involves selecting candidate nipple prosthetic devices wherein the nipple and areola portion are made of natural loosely woven fibrous material and the nipple portion is of a substantially cylindrical shape and is solid. Likewise, selecting the first and subsequent candidate nipple prosthetic devices involves selecting candidate nipple prosthetic devices wherein the nipple portion and areola portion are made of cotton. In addition, selecting the first and subsequent candidate nipple prosthetic devices involves selecting candidate nipple prosthetic devices wherein the nipple portion and the areola portion are made of a breathable microporous water-resistant polymer material.

In addition, selecting the first and subsequent candidate nipple prosthetic devices typically may involve selecting candidate nipple prosthetic devices that contain a nipple portion and an areola portion that are colored to conform to an actual female human nipple and areola and wherein a color of the nipple portion gradually changes to a color of the areola portion in an area of the areola portion closest to the nipple portion.

Similarly, in certain embodiments, prior to attaching the first and subsequent candidate nipple prosthetic devices, the first and subsequent candidate nipple prosthetic devices are peeled off a backing that the adhesive layer of said first and subsequent nipple prosthetic device sticks to during storage.

In certain embodiments, attaching the first and subsequent candidate nipple prosthetic devices involves attaching candidate nipple prosthetic devices wherein the adhesive layer is adjacent to an entire inside surface of the areola portion. In certain embodiments, attaching the first and subsequent candidate nipple prosthetic devices involves attaching candidate nipple prosthetic devices wherein the adhesive layer is also adjacent to at least a part of an inside surface of the nipple portion. In other embodiments, attaching the first and subsequent candidate nipple prosthetic devices involves attaching candidate nipple prosthetic devices wherein the adhesive layer is also adjacent to an entire inside surface of the areola portion and an entire inside surface of the nipple portion.

It should be understood that while the method of the present invention has been described in terms of the sampling by a female patient of the various embodiments of a nipple prosthetic devices described above, other embodiments of a nipple prosthetic device not yet known and having yet to be designed materials may also be used to carry out the method of the present invention provided such yet to be designed materials are breathable and exhibit the hardy characteristics required as described above. Accordingly, the method of the present invention is not intended to be limited to using the specific variations described above.

It is to be understood that while the method of this invention has been described and illustrated in detail, the above-described embodiments are simply illustrative of the principles of the invention. It is to be understood also that various other modifications and changes may be devised by those skilled in the art which will embody the principles of the invention and fall within the spirit and scope thereof. It is not desired to limit the invention to the exact steps of the method described nor to the use of nipple prosthetic devices having the exact construction and operation shown and described. The spirit and scope of this invention are limited only by the spirit and scope of the following claims.

What is claimed is:

1. A method by which a female patient samples nipple prosthetic devices during a second stage of her breast reconstruction, comprising the steps of
the female patient
(a) selecting a first candidate nipple prosthetic device that has a particular size and color,
(b) attaching the first candidate nipple prosthetic device to her breast that is undergoing reconstruction at an initial location of her choosing by means of an adhesive strong enough so that the first candidate nipple prosthetic device stays on a breast during a wearing of the device but weak enough to permit easy removal of said device,
(c) wearing said first candidate nipple prosthetic device at the initial location for a period of time including in as many real life situations as she desires, said period of time optionally including time gaps of non-use, said first candidate nipple prosthetic device being breathable and water-resistant;
(d) removing the first candidate nipple prosthetic device from the initial location,
(e) repeating steps (b) through (d) for the first candidate nipple prosthetic device at as many other locations on said breast as she desires,
(f) repeating steps (a) through (e) for as many subsequent candidate nipple prosthetic devices having a different size and/or shape from a just previously sampled candidate nipple prosthetic device as desired until the female patient satisfactorily decides on a final size, color and location of a desired nipple.

2. The method of claim 1, wherein selecting the first and subsequent candidate nipple prosthetic devices involves selecting candidate nipple prosthetic devices that contain a nipple portion and an areola portion.

3. The method of claim 2, wherein attaching the first and subsequent candidate nipple prosthetic devices involves attaching candidate nipple prosthetic devices wherein the adhesive layer covers an area adjacent to and in contact with at least part of an inside surface of the candidate nipple prosthetic device sufficient to adhere said candidate nipple prosthetic device to the female breast, the areola portion of the candidate nipple prosthetic device surrounding said nipple portion of said candidate nipple prosthetic device and having a concave shape that conforms to an outer contour of a female human breast, said nipple portion aligned with said areola portion so that upon attachment of the candidate nipple prosthetic device to said breast, said nipple portion projecting outwardly from an outer surface of said breast.

4. The method of claim 2, wherein selecting the first and subsequent candidate nipple prosthetic devices involves selecting candidate nipple prosthetic devices wherein the nipple and areola portion are made of natural loosely woven fibrous material and the nipple portion is of a substantially cylindrical shape and is solid.

5. The method of claim 2, wherein selecting the first and subsequent candidate nipple prosthetic devices involves selecting candidate nipple prosthetic devices wherein the nipple portion and areola portion are made of cotton.

6. The method of claim 2, wherein selecting the first and subsequent candidate nipple prosthetic devices involves selecting candidate nipple prosthetic devices wherein the nipple portion and the areola portion are made of a breathable microporous water-resistant polymer material.

7. The method of claim 2, wherein selecting the first and subsequent candidate nipple prosthetic devices involves selecting candidate nipple prosthetic devices that contain a nipple portion and an areola portion that are colored to conform to an actual female human nipple and areola and wherein a color of the nipple portion gradually changes to a color of the areola portion in an area of the areola portion closest to the nipple portion.

8. The method of claim 3, wherein prior to attaching the first and subsequent candidate nipple prosthetic devices, the first and subsequent candidate nipple prosthetic devices are peeled off a backing that the adhesive layer of said first and subsequent nipple prosthetic device sticks to during storage.

9. The method of claim 3, wherein attaching the first and subsequent candidate nipple prosthetic devices involves attaching candidate nipple prosthetic devices wherein the adhesive layer is adjacent to an entire inside surface of the areola portion.

10. The method of claim 11, wherein attaching the first and subsequent candidate nipple prosthetic devices involves attaching candidate nipple prosthetic devices wherein the adhesive layer is also adjacent to at least a part of an inside surface of the nipple portion.

11. The method of claim 3, wherein attaching the first and subsequent candidate nipple prosthetic devices involves attaching candidate nipple prosthetic devices wherein the adhesive layer is also adjacent to an entire inside surface of the areola portion and an entire inside surface of the nipple portion.

12. The method of claim 1, wherein the step of attaching each candidate nipple prosthetic device to a breast involves attaching that candidate nipple prosthetic device to the breast by means of an adhesive layer forming part of said candidate nipple prosthetic device.

13. A method by which a female patient samples nipple prosthetic devices during a second stage of her breast reconstruction, comprising the steps of
the female patient
(a) selecting a first candidate nipple prosthetic device that has a particular set of physical characteristics,
(b) attaching the first candidate nipple prosthetic device to her breast that is undergoing reconstruction at an initial location of her choosing by means of an adhesive strong enough so that the first candidate nipple prosthetic device stays on a breast during a wearing of the device but weak enough to permit easy removal of said device,
(c) wearing said first candidate nipple prosthetic device at the initial location for a period of time including in as many real life situations as she desires, said period of time optionally including time gaps of non-use, said first candidate nipple prosthetic device being breathable and water-resistant;
(d) removing the first candidate nipple prosthetic device from the initial location,
(e) repeating steps (b) through (d) for the first candidate nipple prosthetic device at as many other locations on said breast as she desires,
(f) repeating steps (a) through (e) for as many subsequent candidate nipple prosthetic devices having a different set of physical characteristics from a just previously sampled candidate nipple prosthetic device as desired until the female patient satisfactorily decides on a final set of physical characteristics and location of a desired nipple.

14. The method of claim 13, wherein selecting the first and subsequent candidate nipple prosthetic devices involves selecting candidate nipple prosthetic devices that contain a nipple portion and an areola portion.

15. The method of claim 14, wherein attaching the first and subsequent candidate nipple prosthetic devices involves attaching candidate nipple prosthetic devices wherein the adhesive layer covers an area adjacent to and in contact with at least part of an inside surface of the candidate nipple prosthetic device sufficient to adhere said candidate nipple prosthetic device to the female breast, the areola portion of the candidate nipple prosthetic device surrounding said nipple portion of said candidate nipple prosthetic device and having a concave shape that conforms to an outer contour of a female human breast, said nipple portion aligned with said areola portion so that upon attachment of the candidate nipple prosthetic device to said breast, said nipple portion projecting outwardly from an outer surface of said breast.

16. The method of claim 14, wherein selecting the first and subsequent candidate nipple prosthetic devices involves selecting candidate nipple prosthetic devices wherein the nipple and areola portion are made of natural loosely woven fibrous material and the nipple portion is of a substantially cylindrical shape and is solid.

17. The method of claim 14, wherein selecting the first and subsequent candidate nipple prosthetic devices involves selecting candidate nipple prosthetic devices wherein the nipple portion and areola portion are made of cotton.

18. The method of claim 14, wherein selecting the first and subsequent candidate nipple prosthetic devices involves selecting candidate nipple prosthetic devices wherein the nipple portion and the areola portion are made of a breathable microporous water-resistant polymer material.

19. The method of claim 14, wherein selecting the first and subsequent candidate nipple prosthetic devices involves selecting candidate nipple prosthetic devices that contain a nipple portion and an areola portion that are colored to conform to an actual female human nipple and areola and wherein a color of the nipple portion gradually changes to a color of the areola portion in an area of the areola portion closest to the nipple portion.

20. The method of claim 15, wherein prior to attaching the first and subsequent candidate nipple prosthetic devices, the first and subsequent candidate nipple prosthetic devices are peeled off a backing that the adhesive layer of said first and subsequent nipple prosthetic device sticks to during storage.

21. The method of claim 15, wherein attaching the first and subsequent candidate nipple prosthetic devices involves attaching candidate nipple prosthetic devices wherein the adhesive layer is adjacent to an entire inside surface of the areola portion.

22. The method of claim 23, wherein attaching the first and subsequent candidate nipple prosthetic devices involves attaching candidate nipple prosthetic devices wherein the adhesive layer is also adjacent to at least a part of an inside surface of the nipple portion.

23. The method of claim 15, wherein attaching the first and subsequent candidate nipple prosthetic devices involves attaching candidate nipple prosthetic devices wherein the adhesive layer is also adjacent to an entire inside surface of the areola portion and an entire inside surface of the nipple portion.

24. The method of claim 13, wherein selecting the first and subsequent candidate nipple prosthetic devices involves selecting candidate nipple prosthetic devices whose set of physical characteristics include size, shape, color, rigidity, weight and/or porosity.

25. The method of claim 13, wherein the step of attaching each candidate nipple prosthetic device to a breast involves attaching that candidate nipple prosthetic device to the breast by means of an adhesive layer forming part of said candidate nipple prosthetic device.
